(19) United States
(12) Patent Application Publication
(10) Pub. No.: US 2012/0282237 A1
(43) Pub. Date: Nov. 8, 2012

(54) USE OF PROTEIN ARGININE METHYLTRANSFERASES IN REGULATING HEMATOPOIETIC DIFFERENTIATION

(76) Inventors: Wey-Jinq LIN, Taipei City (TW); Yuan-I Chang, Taipei City (TW); Wei-Kai Hua, Taipei City (TW); Yi-Chi Hung, New Taipei City (TW); Chih-Jen Kuan, Taipei City (TW); Jiun-Shyang Leou, Hsinchu City (TW); Chao-Ling Yao, Hsinchu City (TW); Shiaw-Min Hwang, Hsinchu City (TW)

(21) Appl. No.: 13/100,019

(22) Filed: May 3, 2011

(51) Int. Cl. A61K 38/45; A61P 7/00; A61K 38/18; C12N 5/078; C12Q 1/48

(52) U.S. Cl. 424/94.5; 435/377; 435/15

(57) ABSTRACT

The present invention relates to the manipulations of expression levels or enzymatic activities of PRMTs differentially regulate hematopoietic differentiation toward megakaryocytes or erythrocytes. The modulation of lineage fates of megakaryocyte-erythrocyte progenitors is useful for various purposes, including particular regulation of hematopoiesis for therapy and manufacture of individual blood cells for transplantation.

### TECHNICAL FIELD OF THE INVENTION

The present invention relates to the regulation of hematopoietic differentiation toward megakaryocytes or erythrocytes. Specifically, the invention relates to the modulation of lineage fate of megakaryocyte-erythrocyte progenitors by manipulating expression levels or enzymatic activities of PRMTs.

### BACKGROUND OF THE INVENTION

Megakaryocytes and erythroid are two kinds of unique blood cells in our bodies and play an important role in physiologic hemostasis. Megakaryocytes are bone marrow cells responsible for the production of thrombocytes (also named as platelets), which are necessary for normal blood clotting (Deutsch and Tomer, Br J Haematol 2006, 134, 453-466; Kaushansky, 2006 in Williams hematology, pp. 1571-1585). Erythroid (also referred to as red blood cells) are the most common type of blood cell and can deliver oxygen (O₂) to the body tissues via blood flow through circulatory system (Cantor and Orkin, Oncogene 2002, 21, 3368-3376; Mountford et al., Br J Haematol 2010, 149, 22-34). Both megakaryocytes and erythroid are differentiated from the same multipotent megakaryocyte-erythroid progenitor (MEP), which is derived from the common myeloid progenitors (CMP) (Akashi et al., Nature 2000, 404, 193-197). Diverse results from many experimental systems clearly indicate that CMP is the daughter cell of pluripotential hematopoietic stem cells (HSCs) with restricted differentiation potentials in myeloid lineages (Orkin, Nat Rev Genet 2000, 1, 57-64).

The importance of megakaryopoiesis for clinical medicine is immediately apparent; morbidity and mortality from bleeding due to moderate to severe thrombocytopenia is a major problem facing a wide range of patients. The origins of thrombocytopenia include both iatrogenic and naturally occurring conditions that are frequently encountered in clinical practice (Kaushansky, Blood 2008, 111, 981-986). Unfortunately, platelet transfusion therapy is less than ideal. At least 30% are associated with one or more complications, usually by immune or cytokine-mediated febrile reactions, but occasionally by bacteremia, graft-versus-host disease, or acute pulmonary injury (Kruskall, N Engl J Med 1997, 337, 1914-1915; Rebulla et al., N Engl J Med 1997, 337, 1870-1875). Moreover, an inadequate platelet response due to HLA alloimmunization occurs in from 10% to 30% of individuals who require repeated platelet transfusions, depending on the nature of their disease (Slichter et al., Blood 2005, 105, 4106-4114). Platelet transfusions are expensive. From these, platelet transfusion is not comfortable for treatment of thrombocytopenia.

The principal regulator of megakaryopoiesis in vivo is thrombopoietin (TPO). Knock-out of TPO or its receptor, c-Mpl, results in severe reduction (>80%) in both megakaryocytes in bone marrow and circulating platelets in vivo (Alexander et al., Blood 1996, 87, 2162-2170; de Sauvage et al., J Exp Med 1996, 183, 651-656; Gurney et al., Science 1994, 265, 1445-1447).

Numerous hematopoietic growth factors have been shown to have partial roles in different aspects of megakaryocyte development. Certain cytokines, including granulocyte-macrophage colony-stimulating factor (GM-CSF), interleukin-3 (IL-3), interleukin-6 (IL-6), interleukin-11 (IL-11), interleukin-12 (IL-12), FMS-like tyrosine kinase (FLT) ligand, fibroblast growth factor (FGF) and erythropoietin (Epo), have been known to stimulate the proliferation of megakaryocytic progenitors (Gordon and Hoffman, Blood 1992, 80,302-307; Kaushansky, 2006, vide supra). Taken together, these pleiotropic haematopoietic growth factors seem to regulate the proliferation of megakaryocytic progenitors, but not megakaryocyte differentiation (Chen et al., Stem Cells 1998, 16 Suppl 2, 31-36; Gainsford et al., Blood 2000, 95, 528-534; Gainsford et al., Blood 1998, 91, 2745-2752; Gordon and Hoffman, 1992, vide supra). By contrast, transforming growth factor-β (TGF-β) and interferon-α (IFN-α) mediate suppressive effects of megakaryopoiesis (Kaushansky, 2006).

Based on the importance of cytokines and growth factors in megakaryopoiesis, recombinant human IL-6 and TPO have been used to clinically treat thrombocytopenia in myelodysplastic syndromes (MDS) (Bryan et al., Semin Hematol 2010, 47, 274-280). Unfortunately, IL-6 has only limited activity to improve platelet counts and exhibits significant toxicity, such as fever, chills, and tachycardia, in MDS patents (Gordon et al., Blood 1995, 85, 3066-3076). Recombinant human TPO (rHuTPO) and its shorter, polyethylene glycol-conjugated form, pegylated recombinant megakaryocyte growth and development factor (PEG-rHuMGDF), stimulate platelet production by inducing the growth of megakaryocyte progenitor cells and increase platelet counts in MDS (Kizaki et al., Br J Haematol 2003, 122, 764-767) as well as other thrombocytopenic conditions, such as idiopathic thrombocytopenic purpura (ITP), virus- or chemotherapy-induced thrombocytopenia (Ikeda and Miyakawa, J Thromb Haemost 2009, 7 Suppl 1, 239-244). However, the long-term use of these recombinant proteins develops specific antibodies against endogenous TPO proteins to neutralize its effect, thus leads to discontinue clinical development (Bryan et al., 2010, vide supra). Despite the side effect with the first generation thrombopoietic growth factors, the clinical effectiveness of these agents has led to the development of a second-generation of c-Mpl agonists that appear to be free from causing neutralizing antibodies against endogenous TPO.

Romiplostim, an Fc-peptide fusion protein, and eltrombopag, a nonpeptide agonist, are used clinically to treat patients with the thrombocytopenia. Comparing to first-generation agonists, romiplostim and eltrombopag do not cause neutralizing antibodies throughout the study. Another concern with these agents is their potential to stimulate the proliferation of leukemic cells (Bryan et al., 2010, vide supra). Other agents, such as hypomethylating agents and immunomodulating agents, are used to treat thrombocytopenia, exhibit the low efficiency in platelet productions and unexpective side effects in patients (Bryan et al., 2010, vide supra). Finding new molecular mechanisms in megakaryopoiesis can contribute to develop new therapy approach for treatment of thrombocytopenia.

Erythroid cell transfusion is a well established cellular therapy for various kinds of patients, because it is a well-characterized single cell suspension that lacks nucleated cells and has a low expression of human leucocyte antigen molecules. The problems of transfusion include insufficiency of supply, transfusion transmitted infections, and the requirement for immunological matching persists (Mountford et al., 2010, vide supra). The group O RhD negative erythrocytes reduce the need for immunological matching and other associated risks for the majority of patients. The possibility of generating large numbers of O RhD negative erythroid is therefore an attractive proposition (Mountford et al., 2010, vide supra).

Although in vitro production of enucleated erythroid from hematopoietic progenitor cells in the absence of feeder cells have been developed (Miharada et al., Nat Biotechnol 2006, 24, 1255-1256), large scale manufacture is impossible due to the limited replication capacity of hematopoietic progenitor cells (Mountford et al., 2010, vide supra). Thus, human embryonic stem cells (hESC) and induced pluripotent stem cells (iPSC) may provide an alternative route to produce large amount of erythrocytes for clinical use (Mountford et al., 2010, vide supra). Finding new molecular mechanisms in erythropoiesis can contribute to develop new insight into generation of erythrocytes for clinical use.

Protein arginine methylation is a common protein posttranslational modification regulating diverse cell functions. Protein arginine methylation is catalyzed by protein arginine methyltransferases (PRMTs), which are a family of bisubstrate enzymes that transfer methyl groups from the substrate S-adenosyl-L-methionine (AdoMet) to nitrogen atoms on the guanidine groups of arginine residues within substrate proteins (Bedford and Clarke, Mol Cell 2009, 33, 1-13; Boisvert et al., Sci STKE 2005, 2005, re2; Krause et al., Pharmacol Ther 2007, 113, 50-87; Lakowski et al., Anal Biochem 2010, 397, 1-11; Lee and Stallcup, Mol Endocrinol 2009, 23, 425-433).

The PRMT1 null embryos die at a very early stage, around embryonic day 6.5 to 7.5 after implantation, which implies that PRMT1 plays an important role in regulation of embryonic development (Pawlak et al., Mol Cell Biol 2000, 20,4859-4869; Yu et al., Mol Cell Biol 2009, 29, 2982-2996). Strong expressions of PRMT1 appear along the midline of the neural plate and in the forming head fold, which are related to development of central nervous system from E7.5 to E9.5 (Pawlak et al., 2000, vide supra). Furthermore, the enzyme activity of PRMT1 is elevated during nerve growth factor (NGF)-induced neuronal differentiation of PC 12 cells (Cimato et al., J Neurosci Res 2002, 67, 435-442), and knockdown of PRMT1 suppresses neurite outgrowth of Neuro2a cells (Miyata et al., Neurosci Lett 2008, 445, 162-165). Besides neuronal differentiation, PRMT1 involves in other cell differentiation. The protein expressions and enzymatic activities of PRMT1 in fetal rat liver are much higher than that in newborn rat liver (Lim et al., Biochim Biophys Acta 2005, 1723, 240-247), but the influences of PRMT1 in liver development is unclear.

Carm1 (PRMT4) knockout animals die shortly after birth because they fail to inflate their lungs and have reduced alveolar air space (Yadav et al., Proc Natl Acad Sci USA 2003, 100, 6464-6468), so these observations suggest that CARM1 is an important regulator of lung development. CARM1 also participates in various cell differentiations. The expression of CARM1 mRNA in fetal skeletal muscle during different developmental stages is gradually increased (Peng et al., Anim Genet 2009, 40, 242-246). Furthermore, CARM1 regulates muscle differentiation through cooperation with GRIP-1 (Chen et al., J Biol Chem 2002, 277,4324-4333) or PCAF (Gao et al., J Cell Biochem 2010, 110, 162-170). In addition to muscle differentiation, CARM1 also involves in regulation of other cell differentiations, such as pulmonary epithelial cells (O'Brien et al., Development 2010, 137, 2147-2156), adipocyte (Kim et al., J Biol Chem 2010, 279, 25339-25344; Kowenz-Leutz et al., EMBO J 2010, 29, 1105-1115; Yadav et al., EMBO Rep 2008, 9, 193-198), neuron (Fujiwara et al., Mol Cell Biol 2006, 26, 2273-2285) and myeloid (Kowenz-Leutz et al., 2010, vide supra).

Like PRMT1 in liver development, the protein expressions and enzymatic activities of PRMT5 in fetal rat liver are much higher than that in newborn rat liver (Lim et al., 2005), but the influence is still unclear. In addition, some evidences also indicate that PRMT5 involves in germ cell development (Eckert et al., BMC Dev Biol 2008, 8, 106; Eguizabal et al., Differentiation 2009, 78, 116-123). PRMT5 also participates in muscle differentiation (Dacwag et al., Mol Cell Biol 2009, 29, 1909-1921; Dacwag et al., Mol Cell Biol 2007, 27, 384-394) and renal tubule formation (Braun et al., Am J Nephrol 2004, 24, 250-257).

PRMTs have been reported to exhibit significant roles in regulation of mammalian development and differentiations, however, roles of PRMTs in modulating the commitment of 2 5 megakaryocyte and erythrocyte differentiation are still unclear.

### SUMMARY OF THE INVENTION

This invention is based on the novel discovery of the role of protein arginine methyltransferases (PRMTs) in modulation of lineage fate of megakaryocyte-erythroid progenitors. Manipulations of expression levels or enzymatic activities of PRMTs differentially regulate hematopoietic differentiation toward megakaryocytes or erythroid.

In one aspect, the present invention features a method for regulating hematopoietic differentiation in megakaryocyte-erythroid progenitors, which comprises modulation of the expression level of at least one protein arginine methyltransferase (PRMT) in a target cell.

In one embodiment of the present invention, the modulation of intracellular PRMT expression level is achieved by delivering the coding DNA, antisense DNA, RNA, short hairpin RNA (shRNA), small interference RNA (siRNA), microRNA or protein of PRMT into cells.

In other embodiments of the present invention, the ectopic expression of PRMT suppresses megakaryocyte differentiation but promotes erythroid differentiation, and the reduction in PRMT expression promotes hematopoietic differentiation toward megakaryocyte lineage but inhibits differentiation toward erythroid lineage. In further embodiment of the present invention, the PRMT is PRMT1.

In yet other embodiments of the present invention, the ectopic expression of PRMT promotes megakaryocyte differentiation but suppresses erythroid differentiation, and the reduction in PRMT expression promotes hematopoietic differentiation toward erythroid lineage but inhibits differentiation toward megakaryocyte lineage. In further embodiment of the present invention, the PRMT is PRMT5 or PRMT6.

In another aspect, this invention features a method for regulating hematopoietic differentiation in megakaryocyte-erythroid progenitors, which comprises modulation of the enzymatic activity of at least one protein arginine methyltransferase (PRMT) in a target cell.

In one embodiment of the present invention, the modulation of PRMT activity is achieved by using a chemical compound or peptide to effect the methylation of PRMT substrates.

In other embodiments of the present invention, the hematopoietic differentiation is regulated by the depletion or inhibition of PRMT activity, wherein the depletion or inhibition of PRMT activity increases megakaryocyte differentiation but decreases erythroid differentiation. In further embodiment of the present invention, the PRMT is PRMT1.

In yet other embodiments of the present invention, the hematopoietic differentiation is regulated by the induction or increase of PRMT activity, wherein the induction or increase of PRMT activity promotes megakaryocyte differentiation but decreases erythroid differentiation. In further embodiment of the present invention, the PRMT is PRMT5 or PRMT6.

In another aspect, this invention features a method for screening a drug or compound for regulating hematopoietic differentiation in megakaryocyte-erythroid progenitors, which comprises: cultivating a cell line as a model for megakaryocyte and erythroid differentiation; contacting the cell culture with a candidate drug or compound under a condition suitable for PRMT expression or catalytic reaction; measuring and comparing the expression levels or enzyme activity of PRMT in the cells with and without the treatment of the candidate drug or compound; and determining the promoting or inhibiting effect of the candidate drug or compound on the PRMT expression or PRMT activity to evaluate as a modulator of hematopoietic differentiation.

In one embodiment of the present invention, the candidate drug or compound is evaluated as a suppressor of megakaryocyte differentiation and an enhancer of erythroid differentiation when an elevated expression level or enzyme activity of PRMT1 is observed after the treatment.

In further embodiment of the present invention, the candidate drug or compound is evaluated as an enhancer of hematopoietic differentiation toward megakaryocyte lineage and an inhibitor of differentiation toward erythroid lineage when a reduction in PRMT 1 expression level or enzyme activity is observed after the treatment.

In another embodiment of the present invention, the candidate drug or compound is evaluated as an enhancer of megakaryocyte differentiation and a suppressor of erythroid differentiation when an elevated expression level or enzyme activity of PRMT5 or PRMT6 is observed after the treatment.

Further, the present invention relates to a method for enhancing erythroid differentiation in a subject suffering from erythropenia, which comprises administering a therapeutically effective amount of erythropoietin (EPO) and PRMT1 to the subject in need thereof, wherein the PRMT1 serves as an agonist of EPO-mediated erythropoiesis.

Other features or advantages of the present invention will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1 shows the effect of ectopic expression of HA-PRMT1 on its enzyme activity in K562 cells. K562 cells were stably transfected with pPCDNA3HA2-PRMT1 plasmids (R2-1 and R2-3) or empty vectors (E3-6). (A) Shows the methyltransferase activity of PRMT1 in cell homogenates assayed with a known substrate hnRNP K and detected by fluorography. (B) Shows the levels of asymmetric dimethylarginine in cell lysates detected by Western blot analysis using a specific antibody (ASYM24). (C) The methyltransferase activity of immunoprecipitated HA-PRMT1 proteins with anti-HA antibodies was assayed with recombinant hnRNP A2 and K as substrates (left panel). Mutant hnRNP proteins (ΔGAR) (right panel, in which the consensus PRMT1 methylation regions were deleted) were not methylated.

FIG. 2 shows the regulatory effect of ectopic expression of HA-PRMT1 on megakaryocyte differentiation of K562 cells. Parental K562, empty vector clone E3-6, and PRMT1-overexpressing clones, R2-1 and R2-3, were treated with PMA (40 nM). Megakaryocyte differentiation was detected by modified Wright-Giemsa staining for cytological changes (A), by phase contrast microscopy for examination of adherent cells with pseudopodia (B), or by flow cytometry for the expression of the specific surface marker CD41 (C). Cells with enlarged and lobed nuclei and microvesicles are marked by arrowheads (A, left panel). Dashed lines in (C) are isotypic controls. All experiments were performed at least three times and data are presented as means±S.E.; * p<0.05; ** p<0.01; *** p<0.005 as compared with K562 parental cells.

FIG. 3 shows the regulatory effect of ectopic expression of HA-PRMT 1 on erythroid differentiation of K562 cells. Various K562 cell clones were treated with Ara-C (A, 1 μM) and sodium butyrate (B, 2 mM) for 96 hr, and measured by qualifying hemoglobin-positive cells with benzidine stain. Stable overexpression of PRMT1 (R-1 and R2-3) significantly enhanced erythroid differentiation. All experiments were performed at least three times and data are presented as means±S.E.; *** p<0.005 as compared with K562 parental cells.

FIG. 4 shows the effect of reduced levels of endogenous PRMT1 on both megakaryocyte and erythroid differentiation of K562 cells. KD-1 and KD-2 cell clones were stably transfected with PRMT1 shRNA. Western analysis using an anti-PRMT1 antibody showed the reduced expression of PRMT1 proteins (A). (B) Megakaryocyte differentiation was analyzed by modified Wright-Giemsa staining after PMA treatment. Knockdown of PRMT1 promoted megakaryocyte differentiation. (C) Results of benzidine staining showed that the reduced levels of endogenous PRMT1 significantly suppressed Ara-C- and NaB-induced erythroid differentiation. Luciferase shRNA (Luc) was used as controls. All experiments were performed at least three times and data are presented as means±S.E.; *** p<0.005 as compared with K562 parental cells or Luc clone.

FIG. 5 shows the effects of inhibition and impairment of methyltransferase activity on cell differentiation. The methyltransferase inhibitor, adenosine dialdehyde (AdOx), was added 8 hours before Ara-C treatment. Inhibition of methylation suppressed Ara-C-induced erythroid differentiation (A). The enzymatically impaired mutant PRMT1G80R, when transiently expressed in K562 cells (B), lost the effect on both megakaryocyte (C) and erythroid (D) differentiations. Ectopic expression of mutant PRMT1 did not increase the methyltransferase activity in cell homogenates when assayed with a known PRMT1 substrate hnRNP K (B, lower panel, Fluorography). All experiments were performed at least three times and data are presented as means±S.E.; *** p<0.005 as compared with K562 parental cells.

FIG. 6 shows the enforced expression of PRMT1 in human CD34⁺ hematopoietic cells modulating hematopoietic differentiation. Recombinant TAT-HA-PRMT1 and TAT-HA-GFP (control) proteins were added to CD34⁺ cells. (A) Western blot using anti-HA antibodies examined the entrance of TAT-fused proteins into cells. Erk2 was used as a loading control. After 15 days of thrombopoietin (TPO) treatment, cells were analyzed for expression of CD41, a characteristic of megakaryocytes, by flow cytometry. A representative result from donor 4 was shown in (B). Elevated PRMT1 protein levels decreased expression of CD41 in all four donors examined (Table1). Erythropoietin (EPO) was used to induce erythroid differentiation in CD34⁺ hematopoietic cells. Elevated PRMT1 protein levels promoted EPO-induced erythroid differentiation as analyzed by expressions of hemoglobin (C, left panel) and glycophorin A (C, right panel).

FIG. 7 shows the summarized results for the effect of other PRMTs on megakaryocyte and erythroid differentiation. (A) Western blot using anti-HA antibodies showed the transient expression of HA-tagged PRMT1, PRMT5 or PRMT6 protein in K562 cells. GAPDH was used as a loading control. Megakaryocyte differentiation was analyzed by modified Wright-Giemsa staining 96 hours after PMA treatment (B). Ara-C-(C) and NaB-(D) induced erythroid differentiation was analyzed by benzidine staining. All experiments were performed at least three times and data are presented as means±S.E.; *** p<0.005.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

To study molecular mechanisms of megakaryocyte differentiation, several cell lines have been used as models for megakaryocyte differentiation. Human chronic myelogenous leukemia (CML) cell line K562, which was established from blast crisis of a 53 year old female CML patient (Lozzio and Lozzio, Blood 1975, 45, 321-334), has been used extensively as a model for megakaryocyte and erythroid differentiation because K562 cells behave similar to pluripotent hematopoietic progenitors. Phobol 12-myristate 13-acetate (PMA) can stimulate megakaryocyte differentiation of K562 cells, and PMA-induced megakaryocyte differentiation of K562 mimics, in part, the physiologic events that take place in the bone marrow, including enlarged cell size, increased adhesive properties, vacuolated cytoplasm, formation of multilobed nucleus due to endomitosis, and acquisition of specific markers of megakaryocytes, such as integrin α_{IIb}β₃ (CD41/CD61). Besides, various reagents, such as hemin, sodium butyrate (NaB) and Ara-C (1-beta-D-arabinofuranosylcytosine) can trigger K562 cells to different toward the erythrocyte lineage by promoting expression of erythrocyte-associated hemoglobin, glycophorin A, and spectrin. Thus far, the human K562 cell is a well-established model for study on both megakaryocyte and erythroid differentiation.

Protein arginine methylation is catalyzed by protein arginine methyltransferases (PRMTs), which are a family of bisubstrate enzymes that transfer methyl groups from the substrate S-adenosyl-L-methionine (AdoMet) to nitrogen atoms on the guanidine groups of arginine residues within substrate proteins. Mammalian PRMTs modify the terminal nitrogen atoms to produce ω- N^{G}-monomethylarginine (MMA) residues as a final product or as an intermediate to the formation of one of two types of dimethylarginine (DMA). PRMTs that produce asymmetric ωN^{G},N^{G}-dimethylarginine (aDMA) residues are classified as type I, and those that generate symmetric ω-N^{G},N′^{G}-dimethylarginine (sDMA) residues are classified as type II. PRMT1 and 6 are type I enzymes, and PRMT5 is a type II enzyme.

So far, most of known arginine methylated proteins belong to type I products (aDMA) (Bedford, J Cell Sci 2007, 120, 4243-4246; Bedford and Richard Mol Cell 2005, 33, 1-13; Boisvert et al., 2005, vide supra). Among PRMTs, PRMT1 is the first identified and is the predominant enzyme, which accounts for about 85% of the type I activity in mammals. These results are consistent with the finding that most of currently known arginine methylated proteins are substrates of PRMT1. PRMTs govern a variety of cellular functions in mammalian cells, such as transcription, RNA proceeding, translation, signal transduction, DNA repair, carcinogenesis and viral pathogenesis.

In this invention, it is disclosed that ectopic expression of HA-PRMT1 in K562 cells suppressed PMA-induced megakaryocyte differentiation, while promoted Ara-C- or sodium butyrate (NaB)-induced erythroid differentiation. Knockdown of PRMT1 by small interference RNA exhibited reversed effects on megakaryocyte and erythroid differentiation. Furthermore, the methyltransferase activity of PRMT1 was shown to be required within regulatory manners in differentiations. On the contrary, both PRMT5 and PRMT6 can promote megakaryocyte differentiation, but suppress erythroid differentiation. These results provide evidence for a novel regulatory role of different kinds of PRMTs in hematopoietic differentiation.

### EXAMPLES

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

The human chronic myelogenous leukemia K562 cell line was from the American Type Culture Collection (ATCC). Cells were cultivated in RPMI 1640 medium supplemented with 10% fetal bovine serum, 100 IU/ml penicillin, and 100 IU/ml streptomycin. Transfection was performed by using Lipofectamine™ 2000 reagent (Invitrogen). Stable clones expressing HA-PRMT1 or HA-PRMT1G80R were selected with G418 (0.5 mg/ml, Calbiochem). Stable clones expressing shRNAs were selected with puromycin (0.5 μg/ml, Calbiochem).

For megakaryocyte differentiation, K562 cells were treated with 40 nM of PMA, (Sigma-Aldrich). Adherent cells with pseudopodia were examined by phase contrast light microscopy. For quantification, cells in suspension or loosely attached were first removed from the culture dish carefully, and adherent cells were then removed by trypsinization, collected, and counted. Cytological changes, including a multilobed nucleus and vacuolated cytoplasm, were examined by modified Wright-Giemsa staining. Three to four hundred cells were examined in each assay.

To analyze the megakaryocyte surface marker CD41, cells were first incubated in 2% bovine serum albumin for 30 min and then with fluorescein isothiocyanate (FITC)-conjugated anti-Plt-1 (CD41) antibodies (1:80, Beckman Coulter) in 1% bovine serum albumin for 30 min; cells were then analyzed by flow cytometry.

For the methylation analysis, the thioredoxin-fused hnRNP proteins were expressed and purified as described previously (Chang et al., Electrophoresis 2010, 31, 3834-3842). Cell homogenates (4 μg) and hnRNP proteins (5 μg) were incubated in the presence of 1.65 μCi of [³H]AdoMet (PerkinElmer Life Sciences) and 25 mM Tris-HCl, pH8.0, in a final volume of 30 μl at 30° C. for 30 min. Reactions were stopped by the addition of SDS sample buffer and then subjected to SDS-PAGE. After staining and de-staining, gels were soaked in the fluorographic enhancer EN³HANCE (PerkinElmer Life Sciences), dried, and then exposed to x-ray film (Kodak) at −70° C. for fluorographic analysis.

The Student's t test was used for statistical analysis. Values are presented as means±S.E. All experiments were performed at least three times. p<0.05 was considered statistically significant.

### Ectopic Expression of HA-PRMT1 Differentially Modulates Megakaryocyte and Erythroid Differentiation of K562 Cells

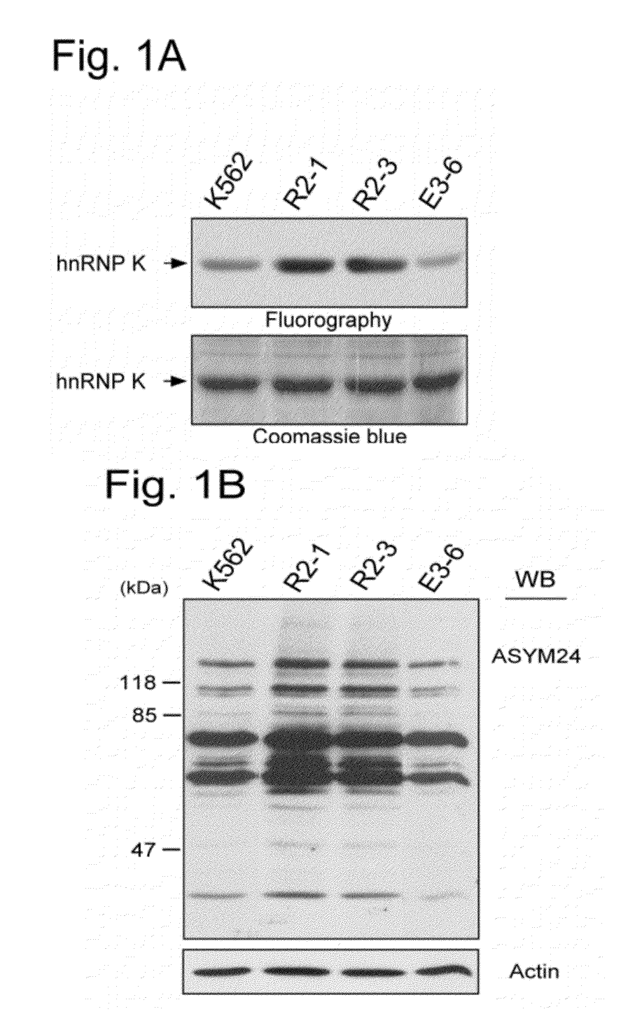

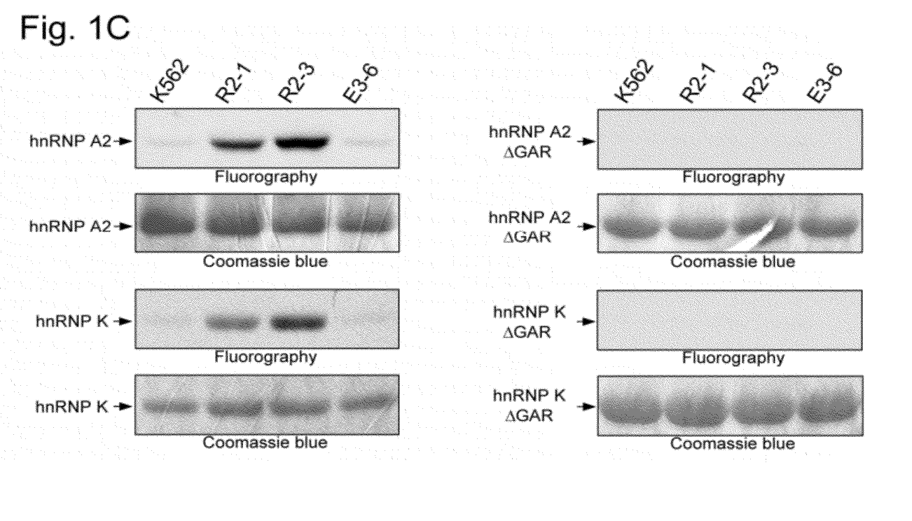

To investigate whether protein arginine methyltransferase 1 (PRMT1) plays a role in megakaryocyte and erythroid differentiation, we established cell clones that stably expressed HA-PRMT1. These stable clones, R2-1 and R2-3, exhibited elevated PRMT1 activity as measured by the methylation of hnRNP K, a known PRMT1 substrate (FIG. 1A) or as measured by the intracellular levels of asymmetric dimethylarginine, a product of PRMT1 catalytic activity (FIG. 1B). The E3-6 cells were an empty vector control that exhibited PRMT1 activity similar to the parental K562 cells. HA-PRMT1 proteins were immunoprecipitated from R2-1 and R2-3 cells, and methylated wild type hnRNP A2 and hnRNP K proteins, but not mutants lacking the methyl acceptor glycine- and arginine-rich (GAR) motifs (FIG. 1C). These results indicated that ectopic PRMT 1 protein was enzymatically active and elevated its activity within R2-1 and R2-3 cell clones.

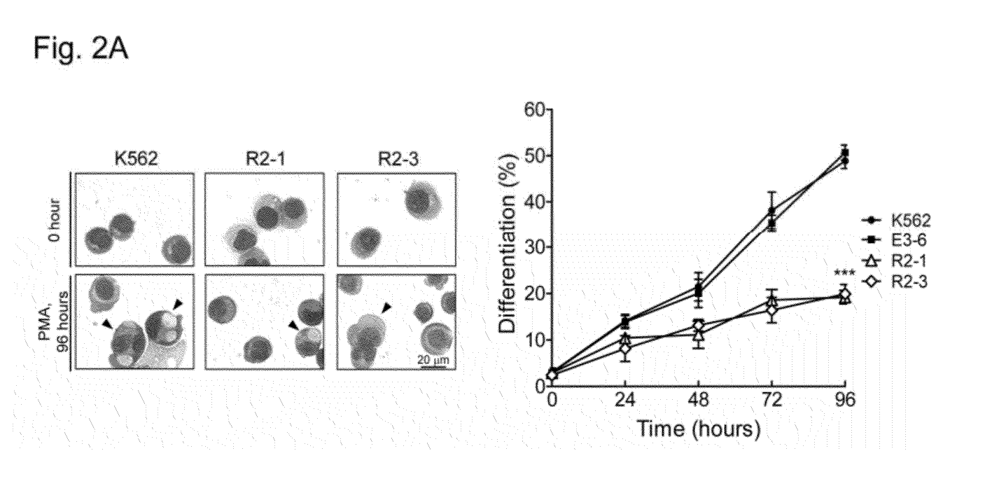

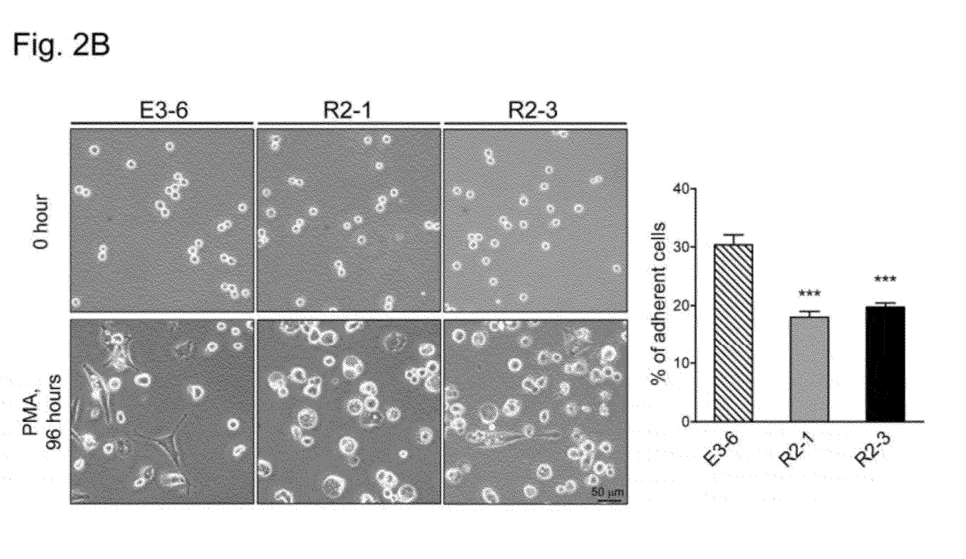

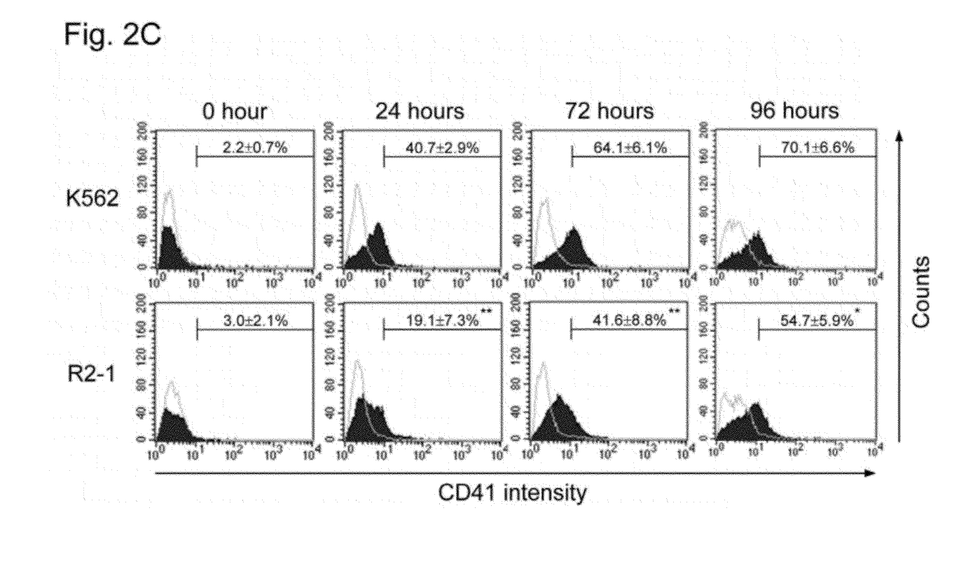

Megakaryopoiesis can be characterized by cytological changes and expression of lineage-specific markers such as CD41. After PMA treatment, K562 cells exhibited enlarged and lobed nuclei and multiple microvesicles, which were readily detected by modified Wright-Giemsa staining (FIG. 2A, left panel, K562). About 50% of the E3-6 cells and the K562 cells exhibited characteristics of megakaryocytes 96 hours after PMA treatment (FIG. 2A, right panel). In addition, we observed an increased number of adherent cells, about 30% at 96 hour (FIG. 2B, E3-6). Only about 20% of R2-1 and R2-3 cells were identified as megakaryocytes by modified Wright-Giemsa staining (FIG. 2A, right panel), indicating that ectopic expression of HA-PRMT1 significantly suppressed differentiation.

The expression of the megakaryocyte-specific marker CD41 on the cell surface was also significantly decreased, from 70% to 55% at 96 hour, in HA-PRMT1-overexpressing cells (FIG. 2C). CD41 is a subunit of integrin α_{IIb}β₃, which is essential for the acquisition of adherent properties. Consistent with reduced CD41 expression in R2-1 and R2-3 cells, fewer adherent cells were observed in these cells, about 17 to 20% at 96 hour (FIG. 2B, R2-1 and R2-3).

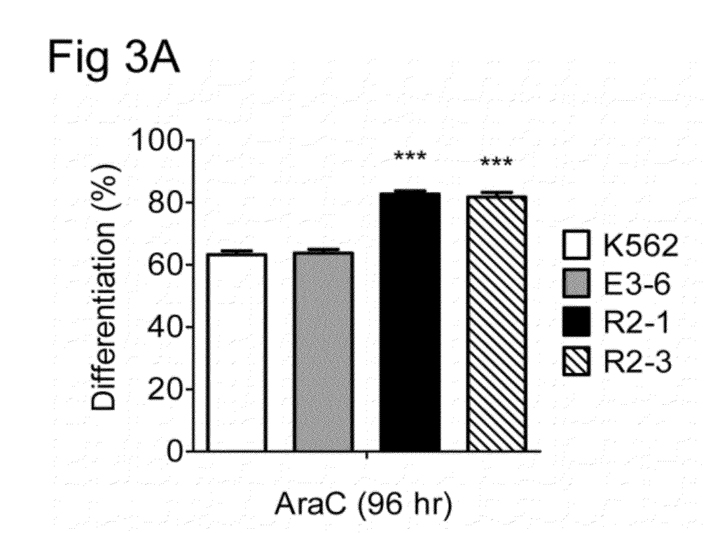

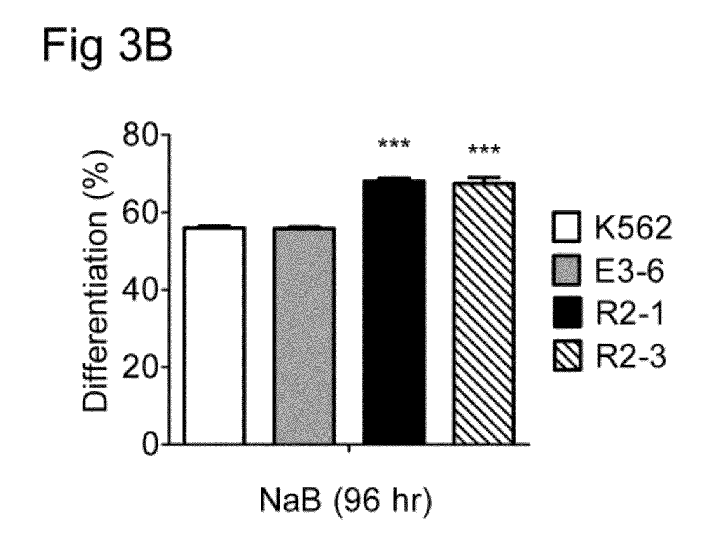

In contrast to the effect of PRMT1 in megakaryocyte differentiation, overexpression of PRMT1 promoted Ara-C-(FIG. 3A) and sodium butyrate (NaB, FIG. 3B)-induced erythroid differentiation, analyzing hemoglobin production by benzidine staining. These results suggested that the enforced expression of PRMT1 differentially modulated megakaryocyte and erythroid lineage commitment in K562 cells.

### Reduced Levels of Endogenous PRMT1 Exhibited Opposite Effects, Compared to Overexpression, in Both Megakaryocyte and Erythroid Differentiation of K562 Cells

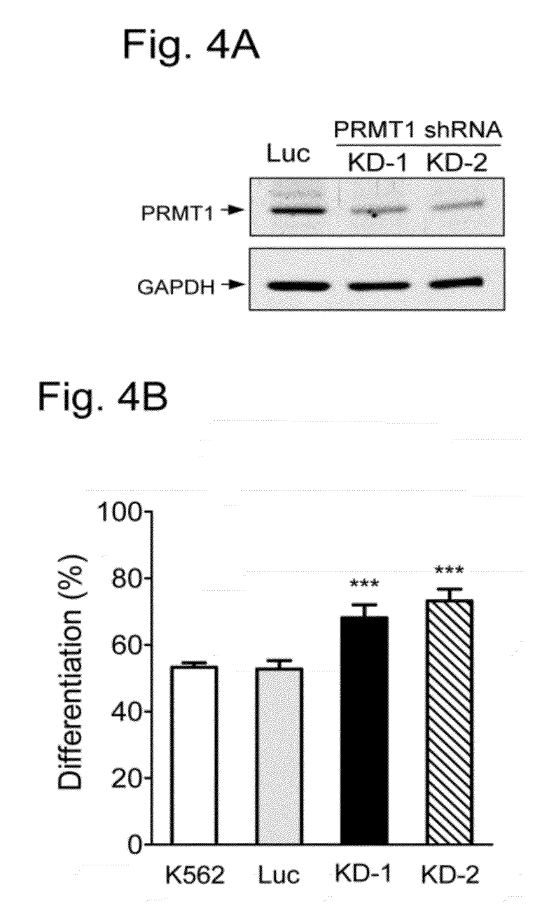

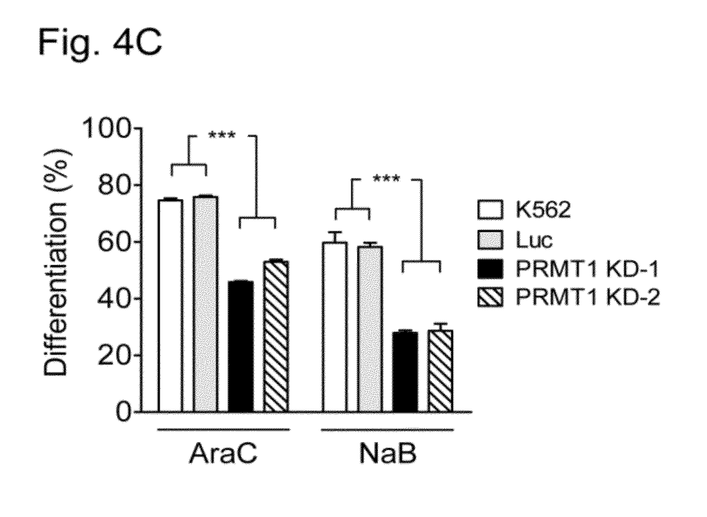

RNA interference was used to investigate whether endogenous PRMT1 plays a role in megakaryocyte and erythroid differentiation. Cell clones that stably expressed PRMT1 shRNA were selected. The protein levels of endogenous PRMT1 in stable clones 1 and 2 (KD-1 and -2) were significantly reduced (FIG. 4A). Upon PMA stimulation, these knockdown clones exhibited significantly higher levels of megakaryocyte differentiation (˜70% as compared to ˜50%; FIG. 4B). However, reduction of PRMT1 significantly decreased Ara-C- and NaB-induced erythroid differentiation (FIG. 4C). These results are consistent with observations from ectopic expression of HA-PRMT1 (FIGS. 2 and 3). Luciferase shRNA (Luc) was used as a control. Taken together, these results suggest that the modulation of PRMT1 expression levels differentially regulate lineage fates of megakaryocyte and erythroid in K562 cells.

### Activity of PRMT1 is Required for its Role in Differentiations

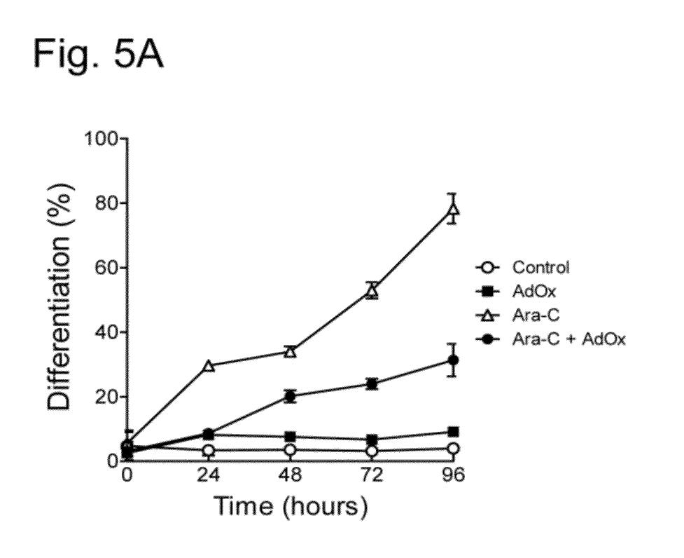

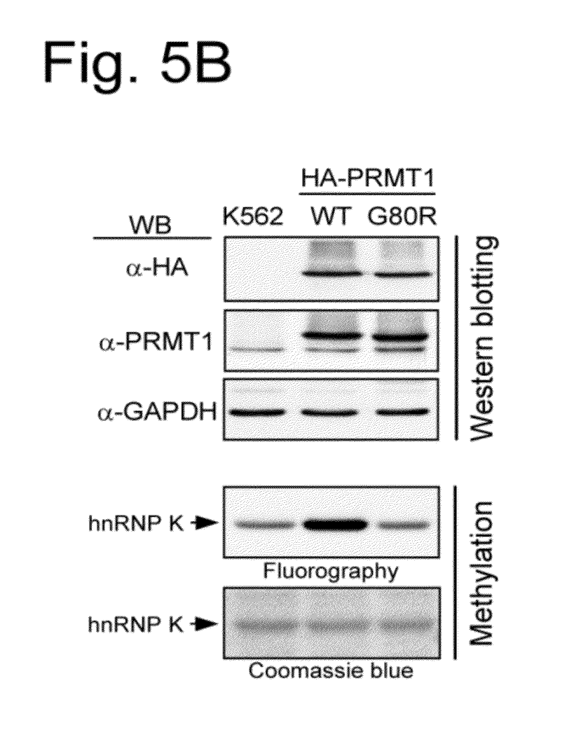

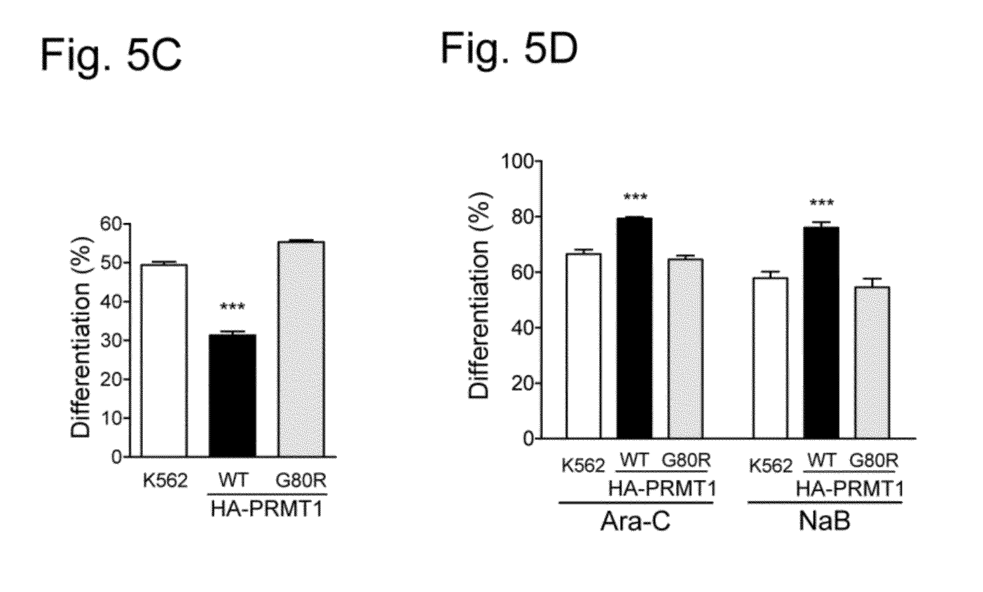

In order to test whether activity of PRMT1 participates in regulation of differentiation, adenosine dialdehyde (AdOx), an inhibitor of S-adenosylhomocysteine hydrolase, which indirectly suppresses methylation due to accumulation of products, was used to treat K562 cells. Inhibition of methylation by AdOx dramatically suppressed Ara-C-induced erythrocytic differentiation, while AdOx alone did not influence differentiation (FIG. 5A). A single amino acid mutation (G80R) in the conserved SAM binding domain of PRMT1 with impaired methyltransferase activity (Kwak et al., Mol. Cell 2003, 11, 1055-1066), HA-PRMT1G80R, was transiently expressed in K562 cells (FIG. 5B). Transient expression of mutant PRMT1 G80R did not suppress PMA-induced megakaryocyte differentiation (FIG. 5C); while decreased Ara-C- and NaB-induced erythroid differentiation (FIG. 5D).

The PRMT1 methyltransferase activity did not affected in PRMT1G80R-overexpressing cell homogenates, comparing to parental cells (FIG. 5B). In contrast, overexpression of wild type PRMT1 significantly enhanced methyltransferase activity (FIG. 5B). Expression levels of these two ectopic proteins were similar as detected by Western blot analysis (FIG. 5B). These results suggest that the methyltransferase activity of PRMT1 is essential for its effect on megakaryocyte and erythroid differentiation, and modulation of its activity can regulate cell fates toward different lineages.

### Modulations of Cell Fates of CD34⁺ Hematopoietic Cells Toward Megakaryocyte and Erythroid Differentiation by PRMT1

To investigate the effect of PRMT1 in more physiologically relevant conditions, TAT-HA-PRMT1 was introduced into human CD34⁺ hematopoietic cells. CD34⁺ cells were derived from human umbilical cord blood with consent from the mother and were collected and processed according to governmental regulations (“Guidelines for Collection and Use of Human Specimens for Research.” Department of Health, Taiwan). Isolation and expansion of CD34⁺ cells were performed as described previously (Yao, et al., Stem Cells Dev. 2006, 15, 70-78).

Briefly, the CD34⁺ cells were purified with CD34 microbeads by a Miltenyi VarioMACS device (Miltenyi Biotec) and cultivated in serum-free Iscove's modified Dulbecco's medium (IMDM) (HyClone) supplemented with serum substitutes (1.5 g/liter bovine serum albumin, 4.4 μg/ml insulin, 60 μg/ml transferrin, and 25.9 μM 2-mercaptoethanol) and a cytokine mixture (8.5 ng/ml TPO, 4.1 ng/ml interleukin-3, 15 ng/ml stem cell factor, 6.7 ng/ml flk2/flt3 ligand, 0.8 ng/ml interleukin-6, 3.2 ng/ml granulocyte colony-stimulating factor, and 1.3 ng/ml granulocyte-macrophage colony-stimulating factor) for 6 to 7 days. To induce megakaryocyte differentiation, the expanded CD34⁺ cells were cultivated (5×10⁴cells/ml) in media described above without cytokine mixture for 6 h. TPO (100 ng/ml) was added to induce differentiation. Expression of CD41 was analyzed by flow cytometry 15 days after TPO stimulation.

Recombinant TAT-fused HA-PRMT1 and HA-GFP (as a control) proteins were expressed in Escherichia coli and purified using Ni⁺-nitrilotriacetic acid-agarose (Qiagen). Endotoxins were removed using Detoxi-Gel™ endotoxin removing gel (Pierce) according to the manufacturer's instructions. For differentiation study, recombinant TAT-fused proteins were added to CD34⁺ cells 6 h before TPO stimulation and again at the time of TPO addition.

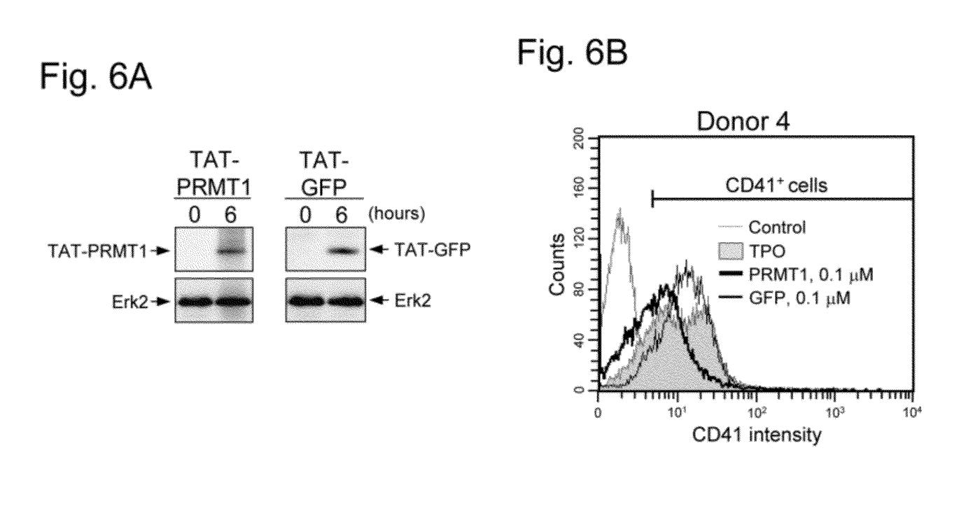

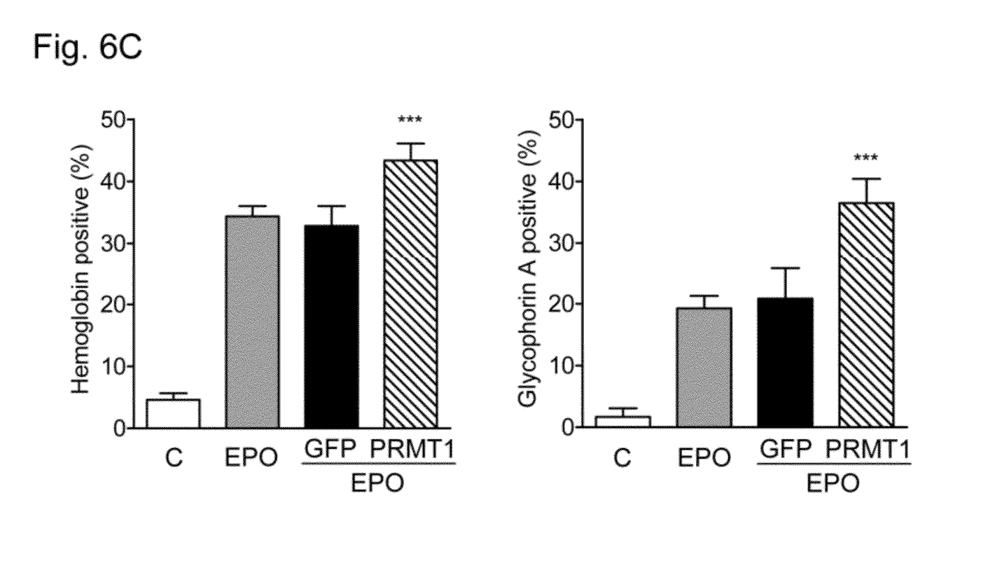

Both the recombinant TAT-conjugated HA-PRMT1 protein and the HA-GFP control protein were detected in cells by Western blot analysis (FIG. 6A). Erk was used as a loading control. Thrombopoietin (TPO) induces CD34⁺ hematopoietic cells to undergo differentiation towards the megakaryocyte lineage as detected by the expression of the specific surface marker CD41. Flow cytometric analysis of donor 4 is shown in FIG. 6B. TPO-induced CD41 expression ranged from 45% to 76% in four different donors at day 15 after TPO treatment (Table 1).

Transduction of TAT-PRMT1 proteins significantly suppressed TPO-induce differentiation (23% to 55% with 0.1 μM of TAT-PRMT1; Table 1). The suppression occurred in a dose-dependent manner (Table 1, donor 4). These results suggest that PRMT1 may negatively regulate TPO-induced megakaryocyte differentiation of CD34⁺ hematopoietic cells.

TABLE 1
Elevated PRMT1 suppressed TPO-mediated megakaryocyte
differentiation in human CD34⁺ hematopoietic cells

| | | | PRMT1 | | GFP | |
|---|---|---|---|---|---|---|
| Donor | Control | TPO | 0.05 μM | 0.1 μM | 0.05 μM | 0.1 μM |
| 1 | 1.6% | 62.3% | 49.3% | — | 65.1% | — |
| 2 | 0.7% | 44.7% | — | 22.5% | — | 43.1% |
| 3 | 3.8% | 68.2% | — | 48.6% | — | 61.2% |
| 4 | 1.9% | 75.7% | 60.3% | 54.6% | 81.8% | 87.4% |

In erythrocyte lineage, TAT-HA-PRMT1 promoted EPO-induced erythroid differentiation of CD34⁺ hematopoietic cells by analyzing expressions of hemoglobin (FIG. 6C, left panel) and specific surface protein, glycophorin A (FIG. 6C, right panel). According to the data showed in FIG. 6C, the effect of EPO on erythroid differentiation was enhanced at the presence of PRMT1. It is suggested that PRMT1 is an agonist for EPO-induced erythroid differentiation.

Taken together, these results not only indicated that PRMT1 differentially modulated cell fates of megakaryocyte and erythroid lineage commitment in CD34⁺ hematopoietic cells, but also provided a hit to efficiently stimulate unique blood cells in vitro from hematopoietic stem cells or other multipotent stem cells by manipulation of PRMT1 level or activity.

### Effects of Other PRMTs in Megakaryocyte and Erythroid Differentiations

In human being, the PRMT family contains at least 10 members (Bedford and Clarke, 2009). To test whether other PRMTs play roles in megakaryocyte and erythroid differentiations, we further examined the effects of PRMT5 and PRMT6 by transient transfection.

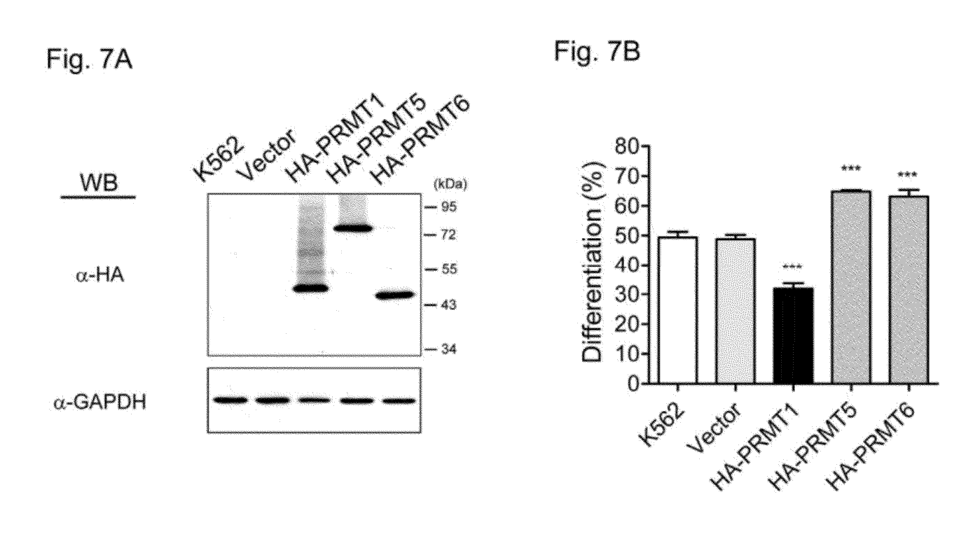

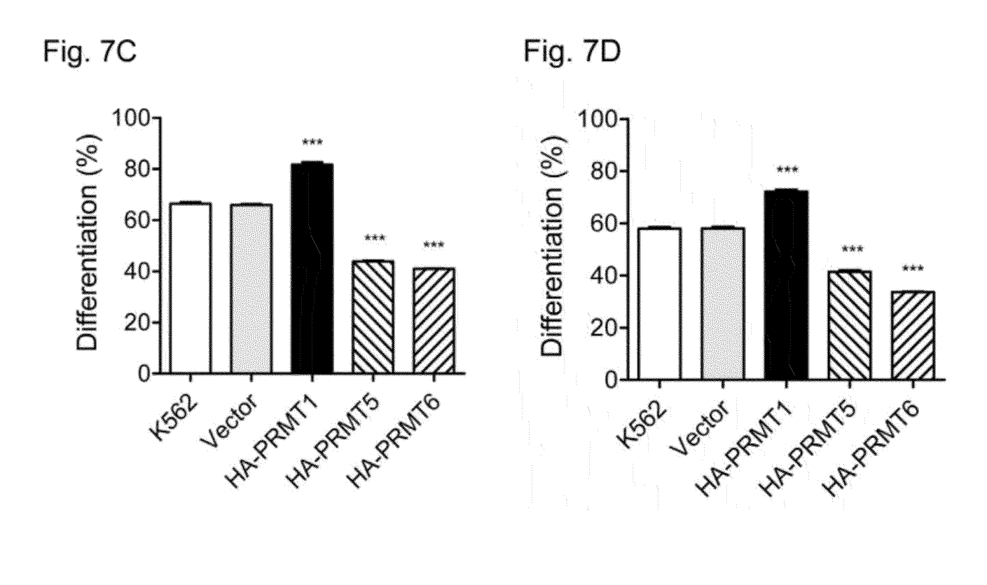

In contrast to PRMT1, ectopic expression of PRMT5 exhibited a stimulatory effect on megakaryocyte differentiation (FIG. 7B). However, PRMT5 suppressed Ara-C- (FIG. 7C) and NaB-induced (FIG. 7D) erythroid differentiation. PRMT6 also increased megakaryocyte differentiation (FIG. 7B), but decreased erythroid differentiation (FIGS. 7C and 7D) in K562 cells. These PRMTs were expressed at a similar level (FIG. 7A). These results suggested a unique role of PRMT family in regulation of megakaryocyte and erythroid differentiation.

In conclusion, ectopic expression of HA-PRMT1 in K562 cells suppressed PMA-induced megakaryocyte differentiation, while stimulated Ara-c or NaB-induced erythroid differentiation. Knockdown of PRMT1 by small interference RNA (siRNA) promoted megakaryocyte differentiation, but suppressed erythroid differentiation. These results suggested that PRMT1 has a significant role in determination of megakaryocyte-erythroid commitment. Furthermore, impairment of the methyltransferase activity of PRMT1, like knockdown of PRMT1, affects megakaryocyte-erythroid commitment. These results provide evidence that modulations of PRMT1 expression levels or methyltransferase activities differentially regulate lineage fates in megakaryocyte-erythrocyte commitment.

These observations were further confirmed in human CD34⁺ hematopoietic cells. Enforced expression of PRMT1 in human CD34⁺^{₊} hematopoietic cells suppresses thrombopoietin (TPO)-induced megakaryocyte differentiation, but stimulate erythropoietin (EPO)-induced erythroid differentiation. These results further supported the role of PRMT1 in regulation of cell fates in megakaryocyte-erythroid commitment.

In contrast to PRMT1, PRMT5 and 6 exhibited different roles to regulate cell fates in megakaryocyte-erythrocyte commitment. In accordance with the present invention, a method of regulating hematopoietic differentiation in megakaryocyte-erythroid progenitors by modulating the expression levels or enzyme activities of PRMTs is provided.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for regulating hematopoietic differentiation of megakaryocyte-erythrocyte progenitors, which comprises modulating the expression level of at least one protein arginine methyltransferase (PRMT) in a target cell.

2. The method of claim 1, wherein the modulation of intracellular PRMT expression level is achieved by delivering the coding DNA, antisense DNA, RNA, short hairpin RNA (shRNA), small interference RNA (siRNA), microRNA or protein of PRMT into cells.

3. The method of claim 1, wherein the hematopoietic differentiation is regulated by the elevated or reduced expression of PRMT.

4. The method of claim 3, wherein the ectopic expression of PRMT suppresses megakaryocyte differentiation but promotes erythroid differentiation, and the reduction in PRMT expression promotes hematopoietic differentiation toward megakaryocyte lineage but inhibits differentiation toward erythroid lineage.

5. The method of claim 4, wherein the PRMT is PRMT1.

6. The method of claim 3, wherein the ectopic expression of PRMT promotes megakaryocyte differentiation but suppresses erythroid differentiation, and the reduction in PRMT expression promotes hematopoietic differentiation toward erythroid lineage but inhibits differentiation toward megakaryocyte lineage.

7. The method of claim 6, wherein the PRMT is PRMT5 or PRMT6.

8. A method for regulating hematopoietic differentiation of megakaryocyte-erythroid progenitors, which comprises modulating the enzymatic activity of at least one protein arginine methyltransferase (PRMT) in a target cell.

9. The method of claim 8, wherein the modulation of PRMT activity is achieved by using a chemical compound or peptide as agonist or antagonist of PRMT.

10. The method of claim 8, wherein the hematopoietic differentiation is regulated by the depletion or inhibition of PRMT activity.

11. The method of claim 10, wherein the depletion or inhibition of PRMT activity increases megakaryocyte differentiation but decreases erythroid differentiation.

12. The method of claim 11, wherein the PRMT is PRMT1.

13. The method of claim 8, wherein the hematopoietic differentiation is regulated by the induction or increase of PRMT activity.

14. The method of claim 13, wherein the induction or increase of PRMT activity increases megakaryocyte differentiation but decreases erythroid differentiation.

15. The method of claim 14, wherein the PRMT is PRMT5 or PRMT6.

16. The method of claim 10, wherein the depletion of PRMT activity is achieved by introducing a PRMT mutant lacking of its enzymatic activity into the target cell.

17. The method of claim 16, wherein the PRMT mutant is introduced by delivering the coding DNA, RNA, or protein of the PRMT mutant.

18. A method for screening a drug or compound for regulating hematopoietic differentiation in megakaryocyte-erythroid progenitors, which comprises:
(1) cultivating a cell line as a model for megakaryocyte and erythroid differentiation;
(2) contacting the cell culture with a candidate drug or compound under a condition for PRMT expression;
(3) measuring and comparing the expression levels of PRMT in the cells with and without the treatment of the candidate drug or compound; and
(4) determining the promoting or inhibiting effect of the candidate drug or compound on the PRMT expression to evaluate as a modulator of hematopoietic differentiation.

19. The method of claim 18, wherein the PRMT is PRMT1.

20. The method of claim 19, wherein the candidate drug or compound is evaluated as a suppressor of megakaryocyte differentiation and an enhancer of erythroid differentiation when an elevated expression of PRMT1 is observed after the treatment.

21. The method of claim 19, wherein the candidate drug or compound is evaluated as an enhancer of hematopoietic differentiation toward megakaryocyte lineage and an inhibitor of differentiation toward erythroid lineage when a reduction in PRMT1 expression is observed after the treatment.

22. The method of claim 18, wherein the PRMT is PRMT5 or PRMT6.

23. The method of claim 22, wherein the candidate drug or compound is evaluated as an enhancer of megakaryocyte differentiation and a suppressor of erythroid differentiation when an elevated expression of PRMT5 or PRMT6 is observed after the treatment.

24. The method of claim 22, wherein the candidate drug or compound is evaluated as an inhibitor of hematopoietic differentiation toward megakaryocyte lineage and an enhancer of differentiation toward erythroid lineage when a reduction in PRMT5 or PRMT6 expression is observed after the treatment.

25. A method for screening a drug or compound for regulating hematopoietic differentiation in megakaryocyte-erythroid progenitors, which comprises:
(1) cultivating a cell line as a model for megakaryocyte and erythroid differentiation;
(2) contacting the cell culture with a candidate drug or compound under a condition for PRMT catalytic activity;
(3) measuring and comparing the enzyme activities of PRMT in the cells with and without the treatment of the candidate drug or compound at the presence of a suitable PRMT substrate; and
(4) determining the promoting or inhibiting effect of the candidate drug or compound on the PRMT activity to evaluate as a modulator of hematopoietic differentiation.

26. The method of claim 25, wherein the PRMT is PRMT1.

27. The method of claim 26, wherein the candidate drug or compound is evaluated as a suppressor of megakaryocyte differentiation and an enhancer of erythroid differentiation when an elevated enzyme activity of PRMT1 is observed after the treatment.

28. The method of claim 26, wherein the candidate drug or compound is evaluated as an enhancer of hematopoietic differentiation toward megakaryocyte lineage and an inhibitor of differentiation toward erythroid lineage when a reduction in PRMT1 enzyme activity is observed after the treatment.

29. The method of claim 25, wherein the PRMT is PRMT5 or PRMT6.

30. The method of claim 29, wherein the candidate drug or compound is evaluated as an enhancer of megakaryocyte differentiation and a suppressor of erythroid differentiation when an elevated enzyme activity of PRMT5 or PRMT6 is observed after the treatment.

31. The method of claim 29, wherein the candidate drug or compound is evaluated as a inhibitor of hematopoietic differentiation toward megakaryocyte lineage and an enhancer of differentiation toward erythroid lineage when a reduction in PRMT5 or PRMT6 enzyme activity is observed after the treatment.

32. A method for enhancing erythroid differentiation in a subject with erythropenia, which comprises administering a therapeutically effective amount of erythropoietin (EPO) and at least one PRMT to the subject in need thereof, wherein the PRMT serves as an agonist of EPO erythropoiesis.

33. The method of claim 32, wherein the PRMT is PRMT1.
